(19) United States
(12) Patent Application Publication
(10) Pub. No.: US 2026/0317293 A1
(43) Pub. Date: Oct. 15, 2026

(54) TICAGRELOR INTERMEDIATE, METHOD FOR PREPARING THE SAME AND METHOD FOR PREPARING TICAGRELOR INTERMEDIATE COMPOUND USING THE SAME

(71) Applicant: Chongqing Puyou Biopharma Co., Ltd., Chongqing (CN)

(72) Inventors: Chenchen QI, Chongqing (CN); Guoxin LAN, Chongqing (CN); Haiyu WEI, Chongqing (CN); Zemi TIAN, Chongqing (CN)

(73) Assignee: Chongqing Puyou Biopharma Co., Ltd., Chongqing (CN)

(21) Appl. No.: 19/481,549

(22) PCT Filed: Jan. 5, 2024
(86) PCT No.: PCT/CN2024/070981

§ 371 Date: Nov. 5, 2025

(30) Foreign Application Priority Data
Oct. 13, 2023 (CN) 202311325712.6

(51) Int. Cl. C07D 317/44; C07C 51/41; C07C 59/255

(52) U.S. Cl. CPC C07D 317/44; C07C 51/412; C07C 59/255

(57) ABSTRACT

This application relates to the field of organic synthesis, especially to the field of organic drug synthesis, and more specifically to a ticagrelor intermediate, a method for preparing the ticagrelor intermediate and a method for preparing a ticagrelor intermediate compound using the ticagrelor intermediate. Using the ticagrelor intermediate can not only reduce synthesis steps, simplify the synthesis process and greatly shorten synthesis time, but also does not require the use of sodium borohydride and palladium/carbon, lowering costs and improving safety, which facilitates industrial use and promotion. In addition, in a subsequent process using the ticagrelor intermediate, a target group is synthesized by means of etherification without ester group reduction, and in a subsequent deprotection process, an active amine is used to effectively prevent inorganic salt residues, and a ticagrelor intermediate compound C can be obtained without washing and extraction. This not only makes the process simpler, but also greatly reduces labor costs, production costs and post-processing costs, and is suitable for industrial production use and promotion.

### TECHNICAL FIELD

The present disclosure relates to the field of organic synthesis, especially to the field of organic drug synthesis, and more specifically to a ticagrelor intermediate, a method for preparing the ticagrelor intermediate and a method for preparing a ticagrelor intermediate compound using the ticagrelor intermediate.

### BACKGROUND

Ticagrelor is an oral selective P2Y12 receptor inhibitor, can prevent adenosine diphosphate-mediated (ADP-mediated) platelet activation and aggregation, and can reduce the probability of cardiovascular disease death, acute coronary syndrome (ACS) in patients with myocardial infarction and stroke, and the historical rate of myocardial infarction (MI).

Ticagrelor has the chemical name (1S,2S,3R,5S)-3-[7-[(1R,2S)-2-(3,4-difluorophenyl)cyclopropylamino]-5-(thiopropyl)-3H-[1,2,3]triazolo[4,5-d]pyrimidin-3-yl]-5-(2-hydroxyethoxy) cyclopentane-1,2-diol, and has the following chemical structure:

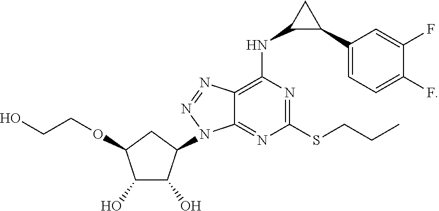

A five-membered ring compound C is an important intermediate compound for synthesizing ticagrelor, and forms a target product ticagrelor by linking with the other two important intermediate compounds, i.e. compound A (pyrimidine ring) and compound C (ternary ring).

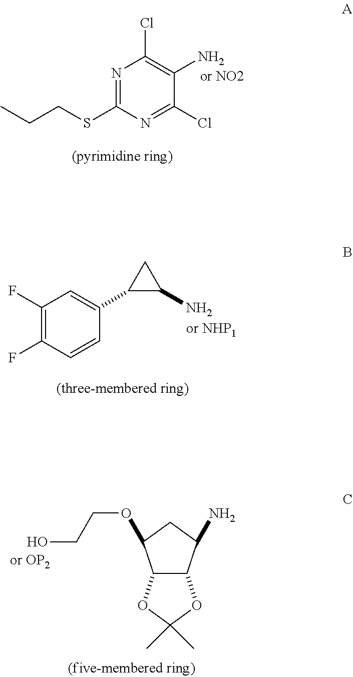

As the compound C has a plurality of chiral centers, the synthesis route and the preparation method of the compound C have always been a hot topic and difficult topic for research of a person skilled in the art.

For example, patent U.S. Pat. No. 7,067,663 reports a preparation method for the intermediate:

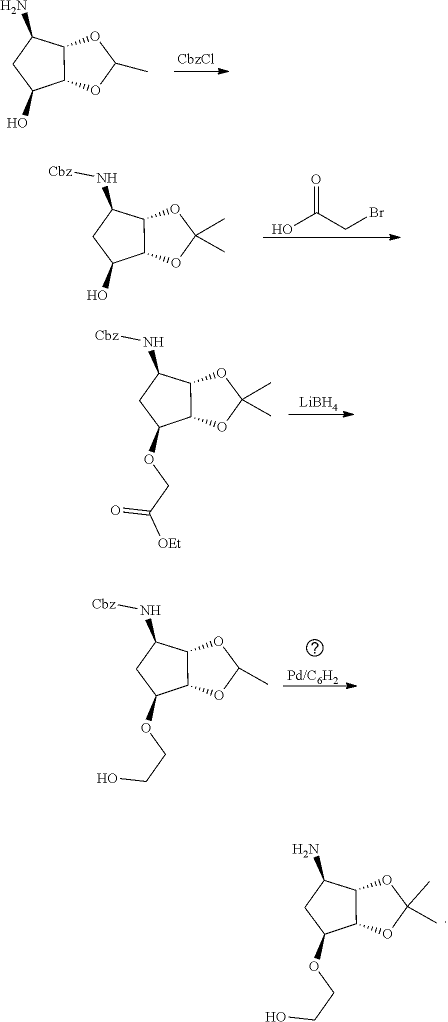

The solution needs expensive lithium borohydride and palladium on carbon, and the entire preparation process has high costs. Moreover, the solution has disadvantages such as many process steps, difficult process operation, and low yield.

For another example, patent WO 2011017108 A also discloses a preparation method for a ticagrelor intermediate, and the synthetic route thereof is as follows:

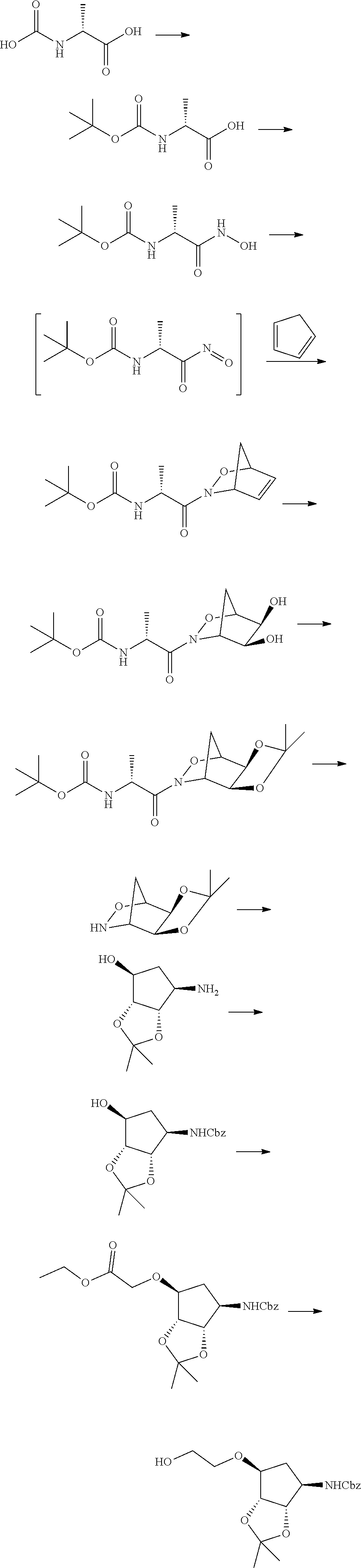

The synthesis route uses D-alanine as a raw material, and an intermediate compound having —Cbz protecting group is prepared through a series of reactions. The reaction has a long route and many steps, and further uses sodium borohydride in the seventh reaction step in the reaction to reduce the substituted —N on the ring, and then further uses palladium on carbon to catalyze the reduction in the eighth reaction step to obtain an NH—O ring-opened compound.

Sodium borohydride and palladium on carbon are not ideal for a person skilled in the art. First of all, sodium borohydride is a strong stimulatory substance, and can produce a strong stimulatory effect on mucosa, upper respiratory tract, eye and skin. After inhalation, spasms, inflammation and edema occur in the larynx and bronchi, which may further lead to chemical pneumonia and pulmonary edema, thereby producing a lethal risk. Furthermore, sodium borohydride itself is unstable and has potential safety risks, so it is not suitable for large-scale use as a raw material in industrial production. Secondly, palladium on carbon has high costs and is difficult to recycle, which results in increased synthetic production costs, and therefore it is not suitable for industrial production. Moreover, it can be determined that in the solution disclosed in this document, the reaction time of the seventh step is 50 minutes, and the pH needs to be adjusted repeatedly so as to obtain a product, and on the basis of the product of the seventh step, the reaction time of the eighth step for ring opening needs 60 minutes, thus the whole process is not only time-consuming but also complicated.

In conclusion, developing a new synthesis method for synthesizing an important intermediate compound C of ticagrelor has always been a focus direction of research and development by a person skilled in the art, and is also a hotspot problem of research in the art.

### SUMMARY

The technical problem to be solved by the present disclosure is to provide a new method for synthesizing an important intermediate compound C of ticagrelor, so as to solve the problems such as long route, many steps, high costs, low safety, and high toxicity existing in the existing synthesis.

In order to solve the described technical problem, the present disclosure discloses a new ticagrelor intermediate having a structural formula as represented by formula 2:

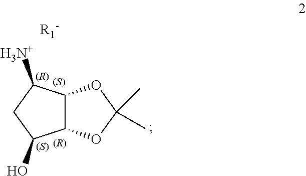

where R₁⁻ is an alkyl acid ion or a sulphonate ion.

Further, the present disclosure discloses a method for preparing the ticagrelor intermediate compound 2. The compound 2 is obtained by a reacting compound 1 with hydrogen gas and an organic acid in the presence of Raney nickel;

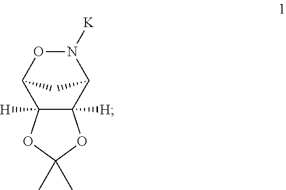

where K is a substituted or unsubstituted benzyl group.

Further, the method for preparing the compound 2 comprises the following steps: (1) adding the compound 1, Raney nickel, a molybdenum powder, an organic acid and a solvent to a reactor, and stirring same; (2) purging air in the system with nitrogen gas; (3) introducing hydrogen gas into the system and pressurizing the system; (4) heating the system to a temperature ranging from 75° C. to 80° C. to initiate a reaction; and (5) after completion of the reaction, filtering to remove the Raney nickel, and concentrating the filtrate under a reduced pressure to dryness to obtain the compound 2.

Optionally, a molar ratio of the compound 1 to the organic acid is 1:1-1:1.2.

Optionally, the organic acid is selected from any one of acetic acid, p-toluenesulfonic acid or pivalic acid, and particularly preferably, the organic acid is pivalic acid. Correspondingly, R₁⁻ is acetate ion, p-toluenesulphonate ion, or pivalate ion, respectively.

Optionally, the solvent is an alcohol solvent, and in a preferred embodiment, the solvent is methanol.

Further, the present disclosure discloses a method for preparing a ticagrelor intermediate compound C using the compound 2, where after the compound 2 is reacted with an anhydride to prepare compound 3, the ticagrelor intermediate compound C is further prepared using the compound 3;

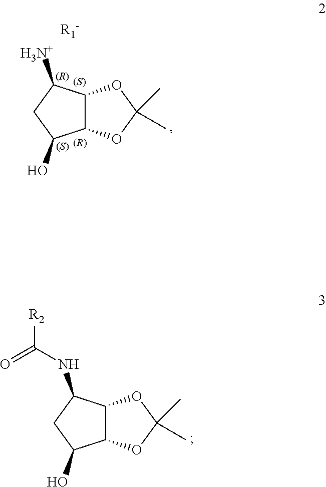

where R₁ is selected from a group consisting of a C1-C5 alkyl group, H, an aryl group, and a substituted aryl group; and R₂ is selected from a group consisting of a C1-C5 alkyl group, an aryl group, and a substituted aryl group.

Optionally, the anhydride is one of acetic anhydride, succinic anhydride, or phthalic anhydride.

Further, the step of preparing the ticagrelor intermediate compound C using the compound 3 includes the following steps:
(a) reacting the compound 3 with an etherification reagent to form compound 4; and
(b) reacting the compound 4 with an active amine compound to obtain compound 5 (compound C).

Optionally, the etherification reagent is selected from one of ethylene carbonate, ethylene sulphate, or ethylene sulphite. Optionally, the etherification reagent is ethylene carbonate. Optionally, a molar ratio of the compound 3 to the etherification reagent is 1:(1-2). Optionally, a reaction temperature in step (a) ranges from 30° C. to 35° C.

Optionally, the active amine is dimethylamine or n-butylamine.

Optionally, the molar ratio of the compound 4 to the active amine is 1:1-1:2.

Optionally, the present disclosure also discloses that the compound 5 is capable of being reacted with tartaric acid to a prepare compound 6.

In an optional technical solution, the molar ratio of the compound 5 to tartaric acid is 1:1-1:2, and specifically may be 1:1.2.

In an optional technical solution, the temperature for reacting the compound 5 with tartaric acid ranges from 40° C. to 55° C., and further, the crystallization temperature ranges from 5° C. to 20° C.

The present disclosure discloses a new synthetic ticagrelor intermediate. Using the ticagrelor intermediate can not only reduce synthesis steps, simplify the synthesis process and greatly shorten synthesis time, but also does not require the use of sodium borohydride and palladium on carbon, lowering costs and improving safety, which facilitates industrial use and promotion. The inventors of the present disclosure surprisingly found that the process can be simplified by using Raney nickel, and the synthesis time can be shortened effectively. In particular, the inventors of the present disclosure creatively found that adding an organic acid can further significantly shorten the reaction time, which produces an unexpected technical effect. In addition, in a subsequent process using the ticagrelor intermediate, a target group is synthesized by means of etherification without ester group reduction, and in a subsequent deprotection process, an active amine is used to effectively prevent inorganic salt residues, and a ticagrelor intermediate compound C can be obtained without washing and extraction. This not only makes the process simpler, but also greatly reduces labor costs, production costs and post-processing costs, and is suitable for industrial production use and promotion.

### DETAILED DESCRIPTION OF THE EMBODIMENTS

The present disclosure will be further illustrated in conjunction with the accompanying drawings and specific embodiments, and it should be understood that the following specific embodiments are merely used for illustrating the present disclosure and not for limiting the scope of the present disclosure.

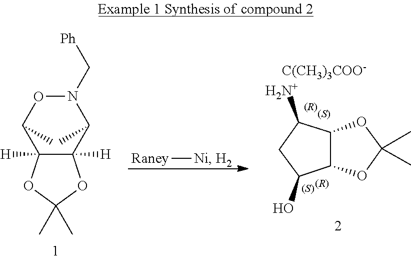

Compound 1 (0.1 mol, 26.13 g), Raney nickel (6.5 g), a molybdenum powder (0.06 g), pivalic acid (0.11 mol, 11.29 g) and methanol (260 ml) were added into a reactor, and stirred until the system was turbid. The air was replaced twice with nitrogen gas, hydrogen gas was introduced into the system, the reactor was pressurized to 1.0 MPa, the system was heated to a temperature ranging from 75° C. to 80° C. for reaction, and the reaction was performed for 8 h.

After the reaction was completed, Raney nickel was filtered off, the filter cake was washed with methanol, and then the methanol phase was combined. Methanol was spin dried under reduced pressure to obtain a light yellow solid, i.e. compound 2 (0.0981 mol, 27.01 g), with a yield of 98.1%.

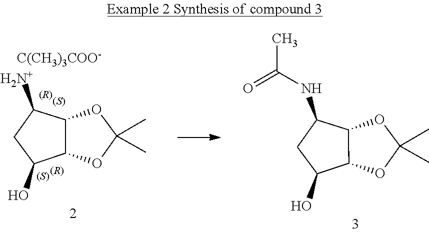

The compound 2 (27.54 g, 0.1 mol) was added into a reactor, and then dichloromethane (250 ml) was added into the reactor. The system was stirred until dissolution, and then the system was cooled to a temperature ranging from 0 to 10° C. Acetic anhydride (10.72 g, 0.105 mol) was added dropwise to the system under a controlled temperature ranging from 0 to 10° C., the addition was completed in 30 min, and the temperature was kept for reaction for 2 h.

After the reaction was completed, a saturated sodium bicarbonate solution was added to adjust the pH to 7-8, the organic layer was separated by standing, the solvent was completely distilled under reduced pressure, and the residue was collected as compound 3 (20.17 g, 0.0937 mol) with a yield of 93.7%.

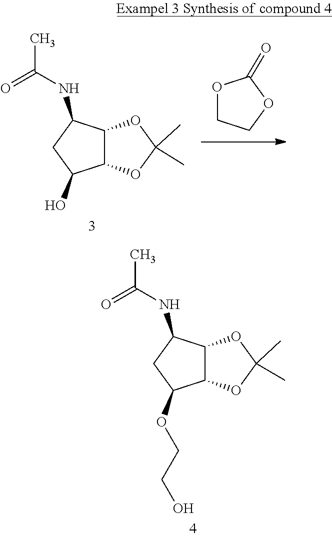

The compound 3 (30 g, 0.14 mol) and ethylene carbonate (14.8 g, 0.168 mol) were added into a reactor, then tetrahydrofuran (150 ml) was added into the reactor, and the system was stirred until dissolution, then potassium tert-butoxide (112.2 g, 0.168 mol) was added into the reactor, the system was heated to a temperature ranging from 30° C. to 35° C., the reaction was carried out for 2 h, then concentrated sulphuric acid (2.8 g, 0.028 mol) was added into the reactor, and the system was stirred for 0.5 min.

After the reaction was finished, the dry solvent was recovered under reduced pressure, and the residue was collected as compound 4 (31.4 g, 0.121 mol) with a yield of 85.9%.

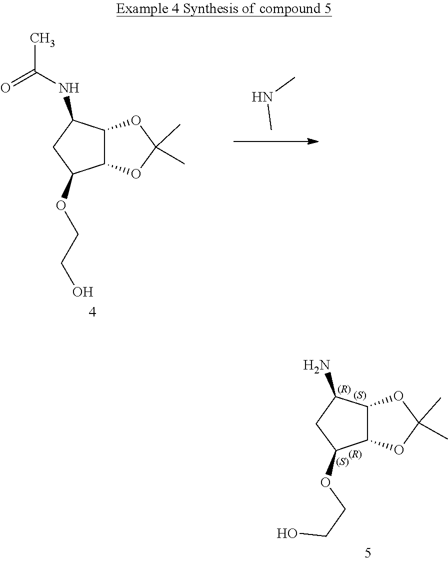

The compound 4 (25.9 g, 0.1 mol) was added into a reactor, then ethanol (200 ml) was added into the reactor, and the system was stirred and mixed, dimethylamine (6.76 g, 0.15 mol) was added dropwise into the reactor, the system was heated to a temperature ranging from 40° C. to 50° C., and the temperature was kept for a reaction for 16 h.

After the reaction was finished, the dry solvent was recovered under reduced pressure to obtain compound 5 (19.99 g, 0.092 mol) with a yield of 92%.

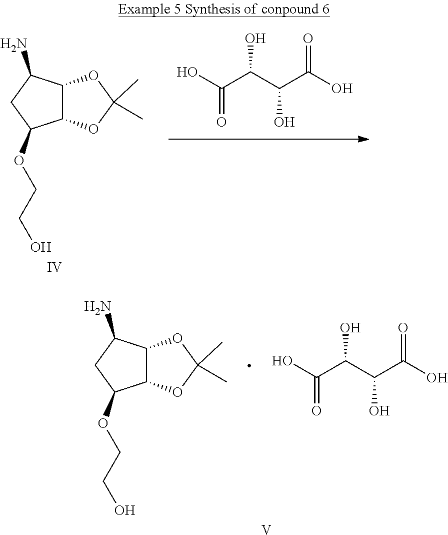

The compound 5 (15.2 g, 0.07 mol) and ethanol (100 ml) were added into a reactor, and the system was stirred until complete dissolution. The system was heated to a temperature ranging from 45° C. to 50° C. At this time, tartaric acid was evenly added in three batches, with an interval of 5 minutes between each batch, and a total amount of tartaric acid was added is 12.6 g (0.084 mol). The temperature was kept at 45-50° C., and stirring was carried out for 1 h.

After the reaction was finished, the system was cooled slowly to a temperature ranging from 30° C. to 40° C. When the temperature reached 30° C., the temperature was further decreased, and a solid was gradually precipitated in the system. Then the system was further cooled to a temperature ranging from 10° C. to 15° C., and the system was stirred for crystallization for 10-12 h.

After crystallization was finished, centrifugation was performed, and the filter cake was washed twice with cold ethanol (30 ml).

After the centrifugation, the wet product was transferred to a vacuum oven and dried at 40-50° C. to obtain compound 6 (22.86 g, 0.0622 mol) with a yield of 88.9% and a liquid phase purity of 99.6%.

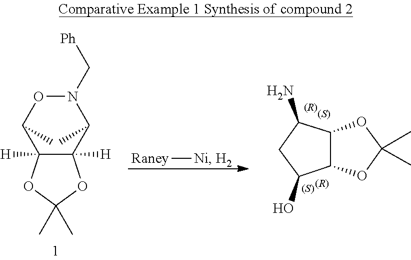

The compound 1 (0.1 mol, 26.13 g), Raney nickel (6.5 g), a molybdenum powder (0.06 g) and methanol (260 ml) were added into a reactor, and the system was stirred until the system was turbid. The air was replaced twice with nitrogen gas, hydrogen gas was introduced into the system, the reactor was pressurized to 1.0 MPa, the system was heated to a temperature ranging from 75° C. to 80° C. for reaction, and the reaction was carried out for 24 h.

After the reaction was completed, Raney nickel was filtered off, the filter cake was washed with methanol, and then the methanol phase was combined. Methanol was spin dried under reduced pressure to obtain a light yellow solid (0.0968 mol, 16.77 g) with a yield of 96.8%.

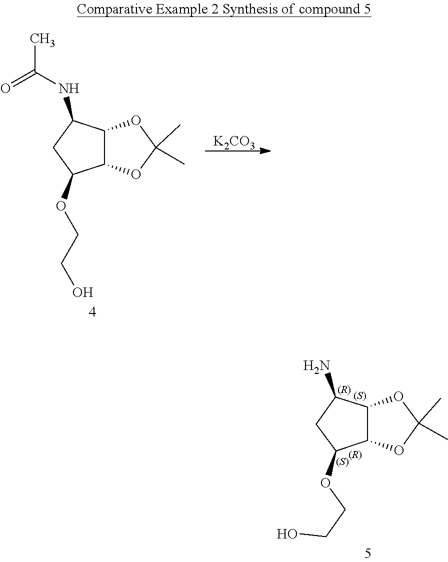

The compound 4 (25.9 g, 0.1 mol) was added to a reactor, then ethanol (200 ml) was added into the reactor and the resulting mixture was mixed while stirring. 100 ml of an aqueous potassium carbonate solution was dropwise added into the reactor, where the content of potassium carbonate is 41.5 g, and the molar concentration of potassium carbonate is 3 mol/L. The system was heated to a temperature ranging from 40° C. to 50° C., and the temperature was kept for a reaction for 16 h.

After the reaction was finished, ethanol in the dry system was recovered under reduced pressure, ethyl acetate (200 ml) was added for extraction twice, the organic layers were combined, and the solvent was evaporated after drying to obtain compound 5 (19.9 g, 0.0916 mol) with a yield of 91.6%.

### Examples 6-8

Other conditions were the same as those in Example 1, except that the molar ratio of the compound 1 to the organic acid and the type of the organic acid were changed. The reaction conditions and yield of Example 1 and Examples 6-8 are shown in Table 1.

TABLE 1
Different conditions and results of Example 1 and Examples 6-8

| | Compound | Complete | Organic | Yield/ |
|---|---|---|---|---|
| Example | 1:organic acid | reaction time | acid species | % |
| 1 | 1:1.1 | 8 | pivalic acid | 98.1 |
| 6 | 1:1 | 8 | pivalic acid | 97.6 |
| 7 | 1:2 | 8 | pivalic acid | 97.7 |
| 8 | 1.1.1 | 10 | acetic acid | 97.6 |

### Examples 9-11

The other conditions were the same as those in Example 2, except that the molar ratio of the compound 2 to the anhydride and the type of the anhydride were changed. The reaction conditions and the yield of Example 2 and Examples 9-11 are shown in Table 2.

TABLE 2
Different conditions and results of Example 2 and Examples 9-11

| | | Anhydride | Yield/ |
|---|---|---|---|
| Example | Compound 2:anhydride | species | % |
| 2 | 1:1.05 | acetic | 93.7 |
| | | anhydride | |
| 9 | 1:1 | acetic | 93.4 |
| | | anhydride | |
| 10 | 1:1.2 | acetic | 92.7 |
| | | anhydride | |
| 11 | 1.1.05 | succinic | 89.8 |
| | | anhydride | |

### Examples 12-14

The other conditions were the same as those in Example 3, except that the molar ratio of the compound 3 to the etherification reagent and the type of the etherification reagent were changed. The reaction conditions and yield of Example 3 and Examples 12-14 are shown in Table 3.

TABLE 3
Different conditions and results of Example 3 and Examples 12-14

| | | Type of | |
|---|---|---|---|
| | Compound 3:etherification | etherification | Yield/ |
| Example | reagent | reagent | % |
| 3 | 1:1.2 | ethylene | 85.9 |
| | | carbonate | |
| 12 | 1:1 | ethylene | 84.8 |
| | | carbonate | |
| 13 | 1:2 | ethylene | 83.5 |
| | | carbonate | |
| 14 | 1:1.2 | vinyl sulfate | 83.8 |

### Examples 15-17

The other conditions were the same as those in Example 4, except that the molar ratio of the compound 4 to active amine and the type of active amine were changed. The reaction conditions and yield of Example 4 and Examples 15-17 are shown in Table 4.

TABLE 4
Different conditions and results of Example 4 and Examples 15-17

| | Compound 4:active | Type of active | |
|---|---|---|---|
| Example | amine | amine | Yield/% |
| 4 | 1:1.5 | dimethylamine | 92 |
| 15 | 1:1 | dimethylamine | 91.2 |
| 16 | 1:2 | dimethylamine | 91.7 |
| 17 | 1:1.5 | n-butylamine | 90.6 |

### Examples 18-23

The other conditions were the same as those in Example 5, except that the molar ratio of the compound 5 to L-tartaric acid and the reaction temperature and crystallization temperature were changed. The reaction conditions and results of Example 5 and Examples 18-23 are shown in Table 5.

TABLE 5
Different conditions and results for Example 5 and Examples 18-23

| | | | Crystalliza- | | |
|---|---|---|---|---|---|
| | Compound | Reaction | tion | | |
| | 5:L-tartaric | temperature/ | temperature/ | Yield/ | Purity/ |
| Example | acid | ° C. | ° C. | % | % |
| 5 | 1:1.2 | 45-50 | 10-15 | 88.9 | 99.6 |
| 18 | 1:1 | 45-50 | 10-15 | 88 | 99.5 |
| 19 | 1:2 | 45-50 | 10-15 | 86.3 | 98.8 |
| 20 | 1:1.2 | 40-45 | 10-15 | 87.1 | 99.1 |
| 21 | 1:1.2 | 50-55 | 10-15 | 88.4 | 98.9 |
| 22 | 1:1.2 | 45-50 | 5-10 | 89.4 | 97.8 |
| 23 | 1:1.2 | 45-50 | 15-20 | 87.3 | 99.5 |

It should be noted that, the foregoing content merely describes the technical idea of the present disclosure, but does not limit the scope of protection of the present disclosure. A person of ordinary skill in the art may further make improvements and modifications without departing from the principle of the present disclosure, and these improvements and modifications all fall within the scope of protection of the claims of the present disclosure.

1. A ticagrelor intermediate, having a structural formula represented by the following formula 1: 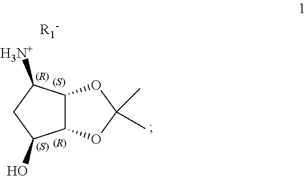
wherein R₁⁻ is an alkyl acid ion or a sulphonate ion.

2. A method for preparing a ticagrelor intermediate having a structural formula represented by the following formula 1: 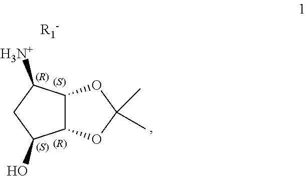
wherein R₁⁻ is an alkyl acid ion or a sulphonate ion;
wherein a reaction of a first compound represented by the following formula 2 with hydrogen gas and an organic acid in the presence of Raney nickel generates the ticagrelor intermediate; 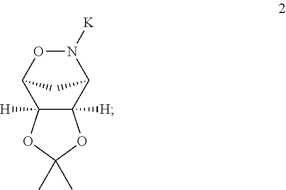
wherein K is a substituted or unsubstituted benzyl group.

3. The method for preparing the ticagrelor intermediate according to claim 2, comprising: adding the first compound, Raney nickel, a molybdenum powder, an organic acid and a solvent to a reactor, and stirring a resulted mixture; purging air in a system consisting of the resulting mixture and the reactor with nitrogen gas; introducing hydrogen gas into the system and pressurizing the system; heating the system to a temperature ranging from 75° C. to 80° C. to initiate a reaction; and after completion of the reaction, filtering to remove the Raney nickel, and concentrating the filtrate under a reduced pressure to dryness to obtain the ticagrelor intermediate.

4. The method for preparing the ticagrelor intermediate according to claim 2, wherein a molar ratio of the first compound to the organic acid is 1:1-1:1.2.

5. The method for preparing the ticagrelor intermediate according to claim 2, wherein the organic acid is selected from any one of acetic acid, p-toluenesulfonic acid or pivalic acid.

6. The method for preparing the ticagrelor intermediate according to claim 3, wherein the solvent is an alcohol solvent.

7. A method for preparing a ticagrelor intermediate compound using a ticagrelor intermediate having a structural formula represented by the following formula 1: 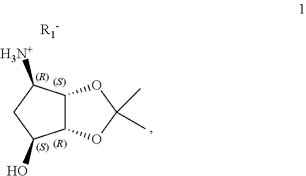
wherein R₁⁻ is an alkyl acid ion or a sulphonate ion;
wherein the method comprises: after the ticagrelor intermediate is reacted with an anhydride to prepare a second compound represented by the following formula 3, preparing the ticagrelor intermediate compound using the second compound; 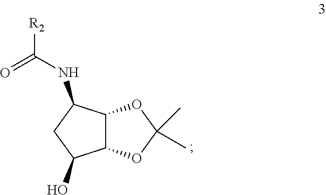
wherein R₂ is selected from a group consisting of a C1-C5 alkyl group, an aryl group, and a substituted aryl group.

8. The method according to claim 7, wherein the anhydride is one of acetic anhydride, succinic anhydride, or phthalic anhydride.

9. The method according to claim 7, wherein the step for preparing the ticagrelor intermediate compound using the second compound comprises:
reacting the second compound with an etherification reagent to form a third compound; and
reacting the third compound with an active amine compound to obtain the ticagrelor intermediate compound.

10. The method according to claim 9, wherein the ticagrelor intermediate compound is capable of being reacted with tartaric acid to prepare a fifth compound;
wherein a method for preparing the fifth compound by reacting the ticagrelor intermediate compound with tartaric acid satisfies at least one of the following conditions:
that a molar ratio of the ticagrelor intermediate compound to tartaric acid is 1:1-1:2;
that a temperature for reacting the ticagrelor intermediate compound with tartaric acid ranges from 40° C. to 55° C.; and
that a crystallization temperature for reacting the ticagrelor intermediate compound with tartaric acid ranges from 5° C. to 20° C.

11. The method according to claim 9, wherein the step for preparing the ticagrelor intermediate compound using the second compound satisfy at least one of the following conditions:
that the etherification reagent is selected from one of ethylene carbonate, ethylene sulphate, or ethylene sulphite;
that the etherification reagent is ethylene carbonate;
that a molar ratio of the second compound to the etherification reagent is 1:1-1:2;
that a reaction temperature in step of reacting the second compound with the etherification reagent ranges from 30° C. to 35° C.;
that the active amine is dimethylamine or n-butylamine; and
that a molar ratio of the third compound to the active amine is 1:1-1:2.

12. The method for preparing the ticagrelor intermediate according to claim 3, wherein a molar ratio of the first compound to the organic acid is 1:1-1:1.2.

13. The method for preparing the ticagrelor intermediate according to claim 3, wherein the organic acid is selected from any one of acetic acid, p-toluenesulfonic acid, or pivalic acid.

14. The method for preparing the ticagrelor intermediate according to claim 13, wherein the organic acid is pivalic acid.

15. The method for preparing the ticagrelor intermediate according to claim 5, wherein the organic acid is pivalic acid.

16. The method for preparing the ticagrelor intermediate according to claim 6, wherein the solvent is methanol.
